(19) United States
(12) Patent Application Publication
(10) Pub. No.: US 2008/0317863 A1
(43) Pub. Date: Dec. 25, 2008

(54) Pharmaceutical Compositions Useful in the Transmucosal Administration of Drugs

(76) Inventors: Christer Nystrom, Stockholm (SE); Nelly Fransen, Uppsala (SE); Erik Bjork, Uppsala (SE)

Correspondence Address: MORGAN LEWIS & BOCKIUS LLP, 1111 PENNSYLVANIA AVENUE NW, WASHINGTON, DC, 20004, US

(21) Appl. No.: 11/884,030

(22) PCT Filed: Feb. 10, 2006
(86) PCT No.: PCT/GB06/00481

§ 371 Date: Feb. 27, 2008

Related U.S. Application Data
(60) Provisional application No. 60/651,210, filed on Feb. 10, 2005.

(51) Int. Cl. A61K 9/50; A61K 39/395; A61K 45/00; A61K 38/21; A61P 43/00

(52) U.S. Cl. 424/491; 424/490; 424/141.1; 424/85.2; 424/85.6; 424/85.7

(57) ABSTRACT

There is provided pharmaceutical compositions in the form of homogeneous interactive mixtures, which compositions comprise a pharmacologically-effective amount of an active ingredient in the form of microparticles of a size between about 0.5 μm and about 10 μm, which particles are attached to the surfaces of larger carrier particles with a size range of between about 10 and about 100 μm. The carrier particle material is preferably bio- and/or mucoadhesive in its nature.

This invention relates to new pharmaceutical compositions for transmucosal administration.

There is a real and growing clinical need for pharmaceutical compositions that provide for fast absorption of drug compounds in order to produce a rapid a therapeutic response. This is particularly the case where a fast acting and/or potent drug compound is to be delivered, for example in the fields of analgesics, antiemetics and sedatives, where such a rapid response is a requirement.

Further, a need exists for further and/or better fast-acting formulations comprising drug compounds that may be administered transmucosally, particularly when such active ingredients are incapable of being delivered perorally due to poor absorption in the gastrointestinal tract.

In order to produce a rapid response, intravenous injection is typically employed, although disadvantages in terms of product fabrication and patient compatibility contribute to the unpopularity of this route of administration.

If appropriate formulations can be devised, nasal administration of drugs may present advantages over other, more typically employed, routes, such as peroral and intravenous administration.

For example, administration of drugs using a nasal spray is convenient and avoids difficulties experienced with peroral administration resulting from the presence of stomach disorders such as nausea.

Moreover, the relatively large available area for mucosal absorption (about 150 cm²) in the nasal cavity is covered with a single epithelial cell layer, over which drugs, including larger hydrophilic molecules that cannot be administered perorally, can pass (see, for example, McMartin et al, J. Pharm. Sci., 76, 535 (1987); Donovan et al, Pharm. Res., 7, 863 (1990) and Fisher et al, J. Pharm. Pharmacol, 44, 550 (1992)). Cells inside the nasal cavity are also highly vascularised, which enables absorbed drug molecules to be transported rapidly into systemic circulation, thereby by-passing first-pass metabolism in the liver.

Today, commercially-available nasal formulations tend to be in the form of liquid sprays. Bioadhesive powder formulations for enhancement of nasal drug uptake have been reported (see, for example, Pereswetoff-Morath, Adv. Drug Deliv. Rev., 29, 185 (1998) and Illum, DDT, 7, 1184 (2002)). Such powders are thought to have a longer residence time in the nasal cavity than liquid formulations. Further, Björk et al (in J. Drug Target 2, 501 (1995)) demonstrated that the swelling of powder particles may induce a temporary opening of the tight junctions between the epithelial cells, which may result in an increase in immediate absorption of active.

Powder formulations for nasal drug delivery are typically in the form of bioadhesive microspheres, which are prepared by dissolving drug and carrier material in a solvent followed by lyophilisation or spray-drying, in order to incorporate the former into the latter (Garcia-Arieta et al, Biol. Pharm. Bull., 24, 1411 (2001)). However, such techniques are physically quite demanding and may therefore present problems for drugs that are inherently unstable (such as peptides), and can give rise to the presence of residual solvent in the final formulation. Moreover, if drug molecules are incorporated within the core of the microspheres, this may lead to a prolonged or delayed release of drug from the resultant formulation, because the release of drug will be dependent on full hydration of the sphere and subsequent diffusion into the epithelium. This is a disadvantage when fast absorption is desired or required.

The avoidance of solvents by employing a technique of co-grinding drug with carrier material has been reported (Provasi et al, Eur. J. Pharm. Biopharm., 40, 223 (1994)). However, in such situations it is still not possible to influence the location of the drug in the formulation.

In this regard, random (i.e. non-interactive; vide infra) mixtures of active ingredients and small lactose carrier particles are presently employed in the delivery of active ingredients to the lung, where they provide a potential alternative to pressurised metered dose inhalers.

U.S. Pat. No. 4,721,709 discloses a formulation for oral use in which drug particles are adsorbed onto the surfaces of carrier particles by a precipitation method. EP 508 255 A1 on the other hand discloses particulate compositions in which peptide drugs are both dispersed homogeneously within carrier particles and on the surfaces of the latter.

There remains, however, a need for an alternative pharmaceutical formulation for transmucosal (e.g. intranasal) delivery of drug compounds, which is capable of providing a prolonged residence time in the relevant cavity, whilst at the same time providing for immediate release and rapid absorption of drug compound.

An “interactive” mixture will be understood by those skilled in the art to denote a mixture in which particles do not appear as single units, as in random mixtures, but rather where smaller particles (of, for example, an active ingredient) are attached to (i.e. adhered to or associated with) the surfaces of larger carrier particles. Such mixtures are characterised by interactive forces (for example van der Waals forces, electrostatic or Coulombic forces, and/or hydrogen bonding) between carrier and drug particles (see, for example, Staniforth, Powder Technol., 45, 73 (1985)). In the final mixture, the interactive forces need to be strong enough to keep the adherent molecules at the carrier surface, in order to create a homogeneous mixture.

In order to obtain a dry powder formulation in the form of an interactive mixture, larger carrier particles must be able to exert enough force to break up agglomerates of smaller drug particles. This ability will primarily be determined by particle density, surface roughness, shape, flowability and, particularly, relative particle sizes. In this respect, the skilled person would expect that, in view of the shear forces that need to be applied during mixing to break up drug particle agglomerates, the smaller the carrier particles, the more difficult it would be to obtain a true interactive mixture.

Surprisingly, we have found that interactive mixtures can be obtained with a high degree of homogeneity with carrier particles of a size of less than 100 μm.

According to a first aspect of the invention, there is provided a pharmaceutical composition in the form of a homogeneous interactive mixture, which composition comprises a pharmacologically-effective amount of an active ingredient in the form of microparticles of a size between about 0.5 μm and about 10 μm, which particles are attached to the surfaces of larger carrier particles with a size range of between about 10 and about 100 μm, which compositions are referred to hereinafter as “the compositions of the invention”.

That homogeneous interactive mixtures can be formed (at all) from primary components with such small relative sizes is indeed surprising. In this respect, there is also provided a process for making a composition of the invention, which process comprises dry mixing carrier particles as defined herein together with particles of active ingredient as defined herein for a sufficient time to provide a homogeneous interactive mixture.

By “homogeneous”, we include that there is a substantially uniform content of active ingredient throughout the powder blend. In other words, if multiple (e.g. at least 30) samples are taken from a composition of the invention (for example as described hereinafter), the measured content of active ingredient that is present as between such samples gives rise to a standard deviation from the mean amount (i.e. the coefficient of variation and/or relative standard deviation) of less than about 10%, such as less than about 8%, for example less than about 5%, particularly less than about 4%, e.g. less than about 3% and preferably less than about 2%. If the majority of the agglomerates of active ingredient are not broken down during mixing, the standard deviation from the mean value will be much higher than these values and, as such, this measure is a direct indicator of the “quality” of a composition in terms of potential dose uniformity.

Alternatively, a “homogenous” interactive mixture may be characterised as a system in which substantially all of the particles of active ingredient are attached to, and/or associated with, the surfaces of the carrier material particles. By “substantially all”, we include that at least 90%, such as at least 95%, for example at least about 98% and preferably at least about 99% of particles of active ingredient are in contact with the surfaces of the carrier particles, as opposed to being “free” (i.e. not associated with the carrier particles) or associated with another part of the carrier particle (i.e. wholly within, or partially penetrating, the carrier particle surface).

Interactive mixture homogeneity may be measured by standard techniques, for example a sampling technique as described hereinafter. Other techniques may include looking directly at a mixture (e.g. by scanning electron microscopy) to determine what proportion of the particles of active ingredient are adhered to, and/or associated with, the carrier particles, as well as blowing an air stream (often with an air velocity in the order of less than 30 litres per minute) over a mixture and analysing the drug fraction that is separated (so testing the amount of drug that is separated from the carriers after actuation from a test actuator).

The term “pharmacologically effective amount” refers to an amount of active ingredient, which is capable of conferring a desired therapeutic effect on a treated patient, whether administered alone or in combination with another active ingredient. Such an effect may be objective (i.e. measurable by some test or marker) or subjective (i.e. the subject gives an indication of, or feels, an effect).

Suitable active ingredients for use in the compositions of the invention include those that may not be administered via the peroral route, for example peptides and peptide hormones (e.g. testosterone), active ingredients that are used in fields where a rapid onset of action is required, for example in the fields of analgesics, antiemetics and sedatives, active ingredients that are highly potent and are therefore typically administered in low doses (for example potent analgesics, such as fentanyl and opioid analgesics, such as morphine) and/or active ingredients that are fast acting (such as sildenafil).

Suitable active ingredients are however not limited by therapeutic category, and may be, for example, analgesics, antiemetics, antiinflammatory agents, anthelmintics, antiarrhythmic agents, antibacterial agents, antiviral agents, anticoagulants, antidepressants, antidiabetics, antiepileptics, antifungal agents, antigout agents, antihypertensive agents, antimalarials, antimigraine agents, antimuscarinic agents, antineoplastic agents, erectile dysfunction improvement agents, immunosuppressants, antiprotozoal agents, antithyroid agents, anxiolytic agents, sedatives, hypnotics, neuroleptics, beta-blockers, calcium channel blockers, cardiac inotropic agents, corticosteroids, decongestants, diuretics, anti parkinsonian agents, gastrointestinal agents, histamine receptor antagonists, keratolytics, lipid regulating agents, antianginal agents, COX-2 inhibitors, leukotriene inhibitors, macrolides, muscle relaxants, nutritional agents, opioid analgesics, potassium channel activators, protease inhibitors, sex hormones, stimulants, muscle relaxants, antiosteoporosis agents, antiobesity agents, cognition enhancers, antiurinary incontinence agents, nutritional oils, antibenign prostate hypertrophy agents, essential fatty acids, non-essential fatty acids, and mixtures thereof.

The active ingredient may also be a cytokine, a peptidomimetic, a peptide, a protein, a toxoid, a serum, an antibody, a vaccine, a nucleoside, a nucleotide, a portion of genetic material, a nucleic acid, or a mixture thereof.

Specific, non-limiting examples of suitable active ingredients include alprazolam, clonazepam, lorazepam, buprenorphine, alfentanil, sufentanil, ramifentanil, granisetron, ramosetron, dolasetron, propofol, tadafinil, vaccines against H5n1 avian influenza and, more particularly, acarbose; acetyl cysteine; acetylcholine chloride; acutretin; acyclovir; alatrofloxacin; albendazole; albuterol; alendronate; alglucerase; amantadine hydrochloride; ambenomium; amifostine; amiloride hydrochloride; aminocaproic acid; aminogluthemide; amiodarone; amlodipine; amphetamine; amphotericin B; antihemophilic factor (human); antihemophilic factor (porcine); antihemophilic factor (recombinant); aprotinin; asparaginase; atenolol; atorvastatin; atovaquone; atracurium besylate; atropine; azithromycin; azithromycin; aztreonam; bacitracin; baclofen; BCG vaccine; becalermin; beclomethasone; belladona; benezepril; benzonatate; bepridil hydrochloride; betamethasone; bicalutanide; bleomycin sulfate; budesonide; bupropion; busulphan; butenafine; calcifediol; calciprotiene; calcitonin human; calcitonin salmon; calcitriol; camptothecan; candesartan; capecitabine; capreomycin sulfate; capsaicin; carbamezepine; carboplatin; carotenes; cefamandole nafate; cefazolin sodium; cefepime hydrochloride; cefixime; cefonicid sodium; cefoperazone; cefotetan disodium; cefotoxime; cefoxitin sodium; ceftizoxime; ceftriaxone; cefuroxime axetil; celecoxib; cephalexin; cephapirin sodium; cerivistatin; cetirizine; chlorpheniramine; cholecalciferol; cholera vaccine; chrionic gonadotropin; cidofovir; cilostazol; cimetidine; cinnarizine; ciprofloxacin; cisapride; cisplatin; cladribine; clarithromycin; clemastine; clidinium bromide; clindamycin andclindamycin derivatives; clomiphene; clomipramine; clondronate; clopidrogel; codeine; coenzyme Q10; colistimethate sodium; colistin sulfate; cortocotropin; cosyntropin; cromalyn sodium; cyclobenzaprine; cyclosporin; cytarabine; daltaperin sodium; danaproid; danazol; dantrolene; deforoxamine; denileulin; diftitox; desmopressin; dexchlopheniramine; diatrizoatemegluamine anddiatrizoate sodium; diclofenac; dicoumarol; dicyclomine; didanosine; digoxin; dihydroepiandrosterone; dihydroergotamine; dihydrotachysterol; diltiazemi; dirithromycin; domase alpha; donepezil; dopamine hydrochloride; doxacurium chloride; doxorubicin; editronate disodium; efavirenz; elanaprilat; enkephalin; enoxacin; enoxaparin sodium; ephedrine; epinephrine; epoetin alpha; eposartan; ergocalciferol; ergotamine; erythromycin; esmol hydrochloride; essential fatty acid sources; etodolac; etoposide; factor IX; famiciclovir; famotidine; felodipine; fenofibrate; fentanyl; fexofenadine; finasteride; flucanazole; fludarabine; fluoxetine; flurbiprofen; fluvastatin; foscarnet sodium; fosphenytion; furazolidone; gabapentin; ganciclovir; gemfibrozil; gentamycin; glibenclamide; glipizide; glucagon; glyburide; glycopyrolate; glymepride; gonadorelin; gonadotropin releasing hormone and synthetic analogs thereof; granulocyte colony stimulating factor; granulocyte-macrophage stimulating factor; grepafloxacin; griseofulvin; growth hormone-bovine; growth hormones-recombinant human; halofantrine; hemophilus B conjugate vaccine; heparin sodium; hepatitis A virus vaccine inactivated; hepatitis B virus vaccine inactivated; ibuprofen; indinavir sulfate; influenza virus vaccine; insulin asparte; insulin detemir; insulin glargine; insulin lispro; insulin NPH; insulin-porcine; insulin-human; interferon alpha; interferon beta; interleukin-2; interleukin-3; ipratropium bromide isofosfamide; irbesartan; irinotecan; isosorbide dinitrate isotreinoin; itraconazole; ivermectin; Japanese encephalitis virus vaccine; ketoconazole; ketorolac; lamivudine; lamotrigine; lanosprazole; leflunomide; leucovorin calcium; leuprolide acetate; levofloxacin; lincomycin and lincomycin derivatives; lisinopril; lobucavir; lomefloxacin; loperamide; loracarbef; loratadine; lovastatin; L-thyroxine; lutein; lycopene; mannitol; measles virus vaccine; medroxyprogesterone; mefepristone; mefloquine; megesterol acetate; meningococcal vaccine; menotropins; mephenzolate bromide; mesalmine; metformin hydrochloride; methadone; methanamine; methotrexate; methoxsalen; methscopolamine; metronidazole; metroprolol; mezocillin sodium; miconazole; midazolam; miglitol; minoxidil; mitoxantrone; mivacurium chloride; montelukast; mumps viral vaccine; nabumetone; nalbuphine; naratriptan; nedocromil sodium; nelfmavir; neostigmine bromide; neostigmine methyl sulfate; neutontin; nicardipine; nicorandil; nifedipine; nilsolidipine; nilutanide; nisoldipine; nitrofurantoin; nizatidine; norfloxacin; octreotide acetate; ofloxacin; olpadronate; omeprazole; ondansetron; oprevelkin; osteradiol; oxaprozin; oxytocin; paclitaxel; pamidronate disodium; pancuronium bromide; paricalcitol; paroxetine; pefloxacin; pentagastrin; pentamidine isethionate; pentazocine; pentostatin; pentoxifylline; periciclovir; phentolamine mesylate; phenylalanine; physostigmine salicylate; pioglitazone; piperacillin sodium; pizofetin; plague vaccine; platelet derived growth factor-human; pneumococcal vaccine polyvalent; poliovirus vaccine inactivated; poliovirus vaccine live (OPV); polymixin B sulfate; pralidoxine chloride; pramlintide; pravastatin; prednisolone; pregabalin; probucol; progesterone; propenthaline bromide; propofenone; pseudoephedrine; pyridostigmine; pyridostigmine bromide; rabeprazole; rabies vaccine; raloxifene; refocoxib; repaglinide; residronate; ribavarin; rifabutine; rifapentine; rimantadine hydrochloride; rimexolone; ritanovir; rizatriptan; rosigiltazone; rotavirus vaccine; salmetrol xinafoate; saquinavir; sertraline; sibutramine; sildenafil (e.g. sildenafil citrate); simvastatin; sincalide; sirolimus; small pox vaccine; solatol; somatostatin; sparfloxacin; spectinomycin; spironolactone; stavudine; streptokinase; streptozocin; sumatriptan; suxamethonium chloride; tacrine; tacrine hydrochloride; tacrolimus; tamoxifen; tamsulosin; targretin; tazarotene; telmisartan; teniposide; terbinafine; terbutaline sulfate; erzosin; tetrahydrocannabinol; thiopeta; tiagabine; ticarcillin; ticlidopine; tiludronate; timolol; tirofibran; tissue type plasminogen activator; tizanidine; TNFR: Fc; TNK-tPA; topiramate; topotecan; toremifene; tramadol; trandolapril; tretinoin; trimetrexate gluconate; troglitazone; trospectinomycin; trovafloxacin; tubocurarine chloride; tumor necrosis factor; typhoid vaccine live; ubidecarenone; urea; urokinase; valaciclovir; valsartan; vancomycin; varicella virus vaccine live; vasopressin and vasopressin derivatives; vecoronium bromide; venlafaxine; vertoporfin; vigabatrin; vinblastin; vincristine; vinorelbine; vitamin A; vitamin B12; vitamin D; vitamin E; vitamin K; warfarin sodium; yellow fever vaccine; zafirlukast; zalcitabine; zanamavir; zidovudine; zileuton; zolandronate; zolmitriptan; zolpidem; zopiclone; and pharmaceutically acceptable salts and derivatives thereof.

In particular, it is envisaged that the active ingredient may comprise a pain management drug such as sumatriptan, zolmitriptan, frovatriptan or dihydroergotamine (migraine) or butorphanol (break through pain); a hormone, such as desmopressin (e.g. desmopressin acetate; diabetes insipidus/polyuria), calcitonin-salmon (hypercalcaemia, Paget's disease), oxytocin (control of labour, bleeding and milk secretion), naferelin and buserelin (endometriosis, CCP), nicotine and vitamin B12 (pernicious anaemia), in addition to alprazolam, clonazepam, lorazepam (anxiolytics), buprenorphine, nalbuphine, alfentanil, sufentanil, ramifentanil (analgesics), granisetron, ramosetron, dolasetron (antiemetics), propofol (sedative, analgesic), tadafinil or sildenafil (erectile dysfunction).

Other specific active ingredients that may be administered by way of compositions of the invention include lobeline, deslorelin, vardenafil, insulin, glucagon, oxycodone, pumactant, apomorphine, lidocaine, dextromethorphane, ketamine, morphine, fentanyl, pramorelin, ondansetron, interferon alpha, interferon beta, scopolamine, vomeropherin, alprazolam, triazolam, midazolam, parathyroid hormone, growth hormone, GHRH, somatostatin, melatonin and several experimental NCEs, and vaccines, such as those for vaccines against H5n1 avian influenza and, more particularly, E coli, streptococcus A, influenza, parainfluenza, RSV, shigella, heliobacter pylori, versinia pestis, AIDS, rabies, periodontitis, and antiarthritic vaccines.

Examples of suitable protein-based active ingredients include blood factors such as Factor VIII (e.g. 80-90 kDa); therapeutic enzymes such as P-glucocerebrosidase (e.g. 60 kDa); hormones such as human growth hormone (somatropin) (e.g. 22.1 kDa); erythropoetin (a glycosylated protein with molecular weight of ca. 30.4 kDa); interferons such as interferon alfacon-1 (e.g. 19.4 kDa), interferon alfa-2b (e.g. 19.2 kDa), peginterferon alfa-2b (e.g. 31 kDa), interferon beta-1a (e.g. 22.5 kDa), interferon beta-1b (e.g. 18.5 kDa) and interferon gamma-1b (e.g. 16.5 kDa); colony stimulating factors such as granulocyte colony stimulating factor (G-CSF, filgrastim) (e.g. 18.8 kDa), pegfilgrastim (e.g. 39kDa) and granulocyte-macrophage colony stimulating factor (GM-CSF, molgramostim, sargramostim) (e.g. 14-20 kDa); interleukins such as interleukin-11 (e.g. 19 kDa), recombinant forms of interleukin-2, such as aldesleulin (e.g. 15.3kDa), and interleukin-1 receptor antagonist (analinra) (e.g. 17.3 kDa); and monoclonal antibodies, such as infliximab.

Most preferred active ingredients include desmopressin, fentanyl, ketamine, buprenorphine and butorphanol.

Any of the above-mentioned active ingredients may be used in combination as required. Moreover, the above active ingredients may be used in free form or, if capable of forming salts, in the form of a salt with a suitable acid or base. If the drugs have a carboxyl group, their esters may be employed. Active ingredients can be used as racemic mixtures or as single enantiomers.

Microparticles of active ingredient are preferably of a particle size of about 0.5 μm (e.g. about 1 μm) to about 8 μm.

Particle sizes are expressed herein as weight based mean diameters. The term “weight based mean diameter” will be understood by the skilled person to include that the average particle size is characterised and defined from a particle size distribution by weight, i.e. a distribution where the existing fraction (relative amount) in each size class is defined as the weight fraction, as obtained e.g. by sieving.

Microparticles of active ingredient may be prepared by standard micronisation techniques, such as grinding, dry milling, wet milling, precipitation, etc.

The amounts of active ingredient that may be employed in compositions of the invention may be determined by the physician, or the skilled person, in relation to what will be most suitable for an individual patient. This is likely to vary with the route of administration, the type and severity of the condition that is to be treated, as well as the age, weight, sex, renal function, hepatic function and response of the particular patient to be treated.

The total amount of active ingredient that may be present in a composition of the invention may be in the range about 0.05 to about 20% (e.g. about 10%) by weight based upon the total weight of the composition. More preferably, compositions of the invention may contain between about 0.07 and about 5% (e.g. about 3%, such as about 2%) by weight of active ingredient, and especially from about 0.1 to about 1%.

The above-mentioned dosages are exemplary of the average case; there can, of course, be individual instances where higher or lower dosage ranges are merited, and such are within the scope of this invention.

We prefer that the carrier particles of the compositions of the invention are bioadhesive and/or mucoadhesive in their nature. In this respect, the compositions of the invention may facilitate the partial or complete adhesion of the active ingredient to a biological surface, such as a mucosal membrane.

International patent applications WO 00/16750 and WO 2004/067004 disclose drug delivery systems for the treatment of acute disorders by e.g. sublingual administration in which the active ingredient is in microparticulate form and is adhered to the surface of larger carrier particles in the presence of a bioadhesive and/or mucoadhesive promoting agent. Formulations comprising carrier particles that consist essentially of bioadhesive and/or mucoadhesive promoting agent, and which are entirely of a size range that is below 100 μm, are not mentioned or suggested anywhere in these documents.

Indeed, to the applicant's knowledge, there has been no previously reported use of an interactive mixture comprising small bioadhesive and/or mucoadhesive carrier particles upon the surfaces of which are adhered smaller particles of active ingredient for direct delivery of the latter to mucosal membranes.

According to a further aspect of the invention there is provided a composition of the invention in which the carrier particles are bioadhesive and/or mucoadhesive in their nature.

It is to be noted that, when the carrier particles are not bioadhesive and/or mucoadhesive in their nature, the coefficient of variation and/or relative standard deviation as defined above is preferably less than about 5%, particularly less than about 4%, e.g. less than about 3% and preferably less than about 2%.

Carrier particles may consist essentially of a bioadhesion and/or mucoadhesion promoting agent. By “consisting essentially” of bioadhesion and/or mucoadhesion promoting agent, we mean that, excluding the possible presence of water (vide infra), the carrier particles comprise at least about 95%, such as at least about 98%, more preferably greater than about 99%, and particularly at least about 99.5% by weight (based on the total weight of the carrier particle) of such an agent. These percentages exclude the presence of trace amounts of water and/or any impurities that may present in such materials, which impurities may arise following the production of such materials, either by a commercial or non-commercial third party supplier, or by a skilled person making a composition of the invention.

The terms “mucoadhesive” and “mucoadhesion” refer to adhesion or adherence of a substance to a mucous membrane within the body, wherein mucous is present on the surface of that membrane (e.g. the membrane is substantially (e.g. >95%) covered by mucous). The terms “bioadhesive” and “bioadhesion” refer to adhesion or adherence of a substance to a biological surface in a more general sense. Biological surfaces as such may include mucous membranes wherein mucous is not present on that surface, and/or surfaces that are not substantially (e.g. <95%) covered by mucous. The skilled person will appreciate that, for example, the expressions “mucoadhesion” and “bioadhesion” may often be used interchangeably. In the context of the present invention, the relevant terms are intended to convey a material that is capable of adhering to a biological surface when placed in contact with that surface (in the presence of mucous or otherwise) in order to enable compositions of the invention to adhere to that surface. Such materials are hereinafter referred to together as “bio/mucoadhesion” promoting agents.

A variety of polymers known in the art can be used as bio/mucoadhesion promoting agents, for example polymeric substances, preferably with an average (weight average) molecular weight above 5,000. It is preferred that such materials are capable of rapid swelling when placed in contact with water and/or, more preferably, mucous, and/or are substantially insoluble in water at room temperature and atmospheric pressure.

Bio/mucoadhesive properties may be routinely determined in a general sense in vitro, for example as described by G. Sala et al in Proceed. Int. Symp. Contr. Release. Bioact. Mat., 16, 420, 1989. Examples of suitable bio/mucoadhesion promoting agents include cellulose derivatives such as hydroxypropylmethyl cellulose (HPMC), hydroxyethyl cellulose (HEC), hydroxypropyl cellulose (HPC), methyl cellulose, ethyl hydroxyethyl cellulose, carboxymethyl cellulose, modified cellulose gum and sodium carboxymethyl cellulose (NaCMC); starch derivatives such as moderately cross-linked starch, modified starch and sodium starch glycolate; acrylic polymers such as carbomer and its derivatives (Polycarbophyl, Carbopol®, etc.); polyvinylpyrrolidone; polyethylene oxide (PEO); chitosan (poly-(D-glucosamine)); natural polymers such as gelatin, sodium alginate, pectin; scleroglucan; xanthan gum; guar gum; poly co-(methylvinyl ether/maleic anhydride); and crosscarmellose (e.g. crosscarmellose sodium). Such polymers may be crosslinked. Combinations of two or more bio/mucoadhesive polymers can also be used.

Suitable commercial sources for representative bio/mucoadhesive polymers include: Carbopol® acrylic copolymer (BF Goodrich Chemical Co, Cleveland, 08, USA); HPMC (Dow Chemical Co., Midland, Mich., USA); NEC (Natrosol; Hercules Inc., Wilmington, Del. USA); HPC (Klucel®; Dow Chemical Co., Midland, Mich., USA); NaCMC (Hercules Inc. Wilmington, Del. USA); PEO (Aldrich Chemicals, USA); sodium alginate (Edward Mandell Co., Inc., Carmel, N.Y., USA); pectin (BF Goodrich Chemical Co., Cleveland, Ohio, USA); crosslinked polyvinylpyrrolidone (Kollidon CL®, BASF, Germany, Polyplasdone XL®, Polyplasdone XL-10® and Polyplasdone INF-10®, ISP Corp., US); Ac-Di-Sol® (modified cellulose gum with a high swellability; FMC Corp., USA); Actigum (Mero-Rousselot-Satia, Baupte, France); Satiaxana (Sanofi BioIndustries, Paris, France); Gantrez® (ISP, Milan, Italy); chitosan (Sigma, St Louis, Miss., USA); and sodium starch glycolate (Primojel®, DMV International BV, Netherlands, Vivastar®, J. Rettenmaier & Söhne GmbH & Co., Germany, Explotab®, Roquette America, US).

Preferred bio/mucoadhesive materials include sodium starch glycolate and crosslinked polyvinylpyrrolidone.

Depending on the type of the bio/mucoadhesion promoting agent used, the rate and intensity of bio/mucoadhesion may be varied.

Suitably, the amount of bio/mucoadhesion promoting agent that is present in a composition of the invention may be in the range about 60.0 to about 99.9% by weight based upon the total weight of the composition. A preferred range is from about 70 to about 99% by weight.

Preferably, carrier particles for use in compositions of the invention are of a size of between about 15 and about 95 μm, such as about 90 μm, and more preferably about 80 μm, for example about 20 and about 65 (such as about 60) μm.

Compositions of the invention may comprise a pharmaceutically acceptable surfactant or wetting agent, which may enhance that hydration of the active ingredient and carrier particles, resulting in faster initiation of both mucoadhesion and dissolution. If present, the surfactant should be provided in finely dispersed form and mixed intimately with the active ingredient. Examples of suitable surfactants include sodium lauryl sulphate, lecithin, polysorbates, bile acid salts and mixtures thereof. If present, the surfactant may comprise between about 0.3 and about 5% by weight based upon the total weight of the composition, and preferably between about 0.5 and about 3% by weight.

Compositions of the invention may be administered as a dry powder, or may directly compressed/compacted into unit dosage forms (e.g. tablets), for administration to mammalian (e.g. human) patients.

In compositions of the invention that are in the form of tablets, a binder and/or disintegrating agent or “disintegrant” may also be employed.

A binder may be defined as a material that is capable of acting as a bond formation enhancer, facilitating the compression of the powder mass into coherent compacts. Suitable binders include cellulose gum and microcrystalline cellulose. If present, binder is preferably employed in an amount of between 0.5 and 20% by weight based upon the total weight of the tablet formulation. A preferred range is from 1 to 15%, such as from about 2.0 to about 12% (e.g. about 10%) by weight.

A disintegrant may be defined as a material that is capable of accelerating to a measurable degree the disintegration/dispersion of a tablet formulation and in particular carrier particles, as defined herein. This may be achieved, for example, by the material being capable of swelling and/or expanding when placed in contact with water and/or mucous (e.g. saliva), thus causing the tablet formulations/carrier particles to disintegrate when so wetted. Suitable disintegrants include cross-linked polyvinylpyrrolidone, carboxymethyl starch and natural starch. If present, disintegrating agent is preferably employed in an amount of between 0.5 and 10% by weight based upon the total weight of the tablet formulation. A preferred range is from 1 to 8%, such as from about 2 to about 7% (e.g. about 5%) by weight.

It will be evident from the list of possible disintegrants provided above that certain materials may function in compositions of the invention in the form of tablets both as bio/mucoadhesion promoting agents and as disintegrating agents. Thus, these functions may both be provided by the same substance or may be provided by different substances.

In compositions of the invention that are in the form of tablets, suitable further additives and/or excipients may also comprise:
(a) lubricants (such as sodium stearyl fumarate or magnesium stearate). When a lubricant is employed it should be used in very small amounts (e.g. up to about 3%, and preferably up to about 2%, by weight based upon the total weight of the tablet formulation);
(b) flavourings (e.g. lemon, menthol or peppermint powder), sweeteners (e.g. neohesperidin) and dyestuffs;
(c) antioxidants, which may be naturally occurring or otherwise (e.g. vitamin C, vitamin E, β-carotene, uric acid, uniquion, SOD, glutathione peroxidase or peroxidase catalase); and/or
(d) other ingredients, such as carrier agents, preservatives and gliding agents.

Wherever the word “about” is employed herein in the context of dimensions (e.g. particle sizes), amounts (e.g. relative amounts of individual constituents in a composition or a component of a composition, and numbers of active particles adhered to carrier particles) and standard deviations, it will be appreciated that such variables are approximate and as such may vary by ±10%, for example ±5% and preferably ±2% (e.g. ±1%) from the numbers specified herein.

Compositions of the invention may be prepared by standard techniques, and using standard equipment, known to the skilled person (see, for example, Lachman et al, “The Theory and Practice of Industrial Pharmacy”, Lea & Febiger, 3^{rd} edition (1986) and “Remington: The Science and Practice of Pharmacy”, Gennaro (ed.), Philadelphia College of Pharmacy & Sciences, 19^{th} edition (1995)).

For example, a suitable grain size fraction of carrier particles is prepared, for example by passing particles comprising such material through a screen or sieve of an appropriate mesh size.

Techniques such as spray drying and surface precipitation may be employed to deposit active ingredient onto the surface of carrier particles. This may be achieved by, for example, techniques such as pipetting, soaking, or rotary evaporation of, a solution of active ingredient onto carrier particles, for example as described hereinafter). Active ingredient may alternatively be dry mixed with carrier particles over a period of time that is sufficiently long to enable appropriate amounts of active ingredient as specified hereinbefore to adhere to the surface of the carrier particles. Standard mixing equipment may be used in this regard. The mixing time period is likely to vary according to the equipment used.

The skilled person will appreciate that whatever the technique employed for manufacture of a composition of the invention, that technique should not change the essential bioadhesive nature of the carrier particles.

If appropriate, other ingredients (e.g. binders/disintegrants and surfactants) may be incorporated by standard mixing as described above for the inclusion of active ingredient.

If a tablet formulation is required, dry powders obtained by mixing may be directly compressed/compacted into unit dosage forms. (See, for example, Pharmaceutical Dosage Forms: Tablets. Volume 1, 2^{nd} Edition, Lieberman et al (eds.), Marcel Dekker, New York and Basel (1989) p. 354-356 and the documents cited therein.) Suitable compacting equipment includes standard tabletting machines, such as the Kilian SP300 or the Korsch EK0.

Irrespective of the foregoing, the composition of the invention should be essentially free (e.g. less than 20% by weight based on the total weight of the formulation) of water. It will be evident to the skilled person that “premature” hydration may dramatically decrease the mucoadhesion promoting properties of a composition and may result in premature dissolution of the active ingredient.

The compositions of the invention may be administered pulmonarily, rectally, to the oral mucosa (e.g. sublingually) or, preferably, intranasally by way of appropriate dosing means known to the skilled person.

The compositions of the invention may be used to treat/prevent diseases/conditions in mammalian patients depending upon the therapeutic agent which is employed. For the particular active ingredients mentioned herein, diseases/conditions which may be mentioned include those against which the active(s) in question is/are known to be effective, and include those specifically listed for the actives in question in Martindale, “The Extra Pharmacopoeia”, 34th Edition, Royal Pharmaceutical Society (2004).

According to a further aspect of the invention, there is provided a method of treatment of a disease, which comprises administration of a composition of the invention to a patient in need of such treatment.

For the avoidance of doubt, by “treatment” we include the therapeutic treatment, as well as the symptomatic treatment, the prophylaxis, or the diagnosis, of a condition.

The compositions of the invention enable the production of dosage forms that are easy and inexpensive to manufacture, and which enable the rapid release and/or a rapid uptake of the active ingredient employed through the mucosa, thus enabling a rapid therapeutic effect.

The compositions of the invention enable such rapid absorption of active ingredient to be achieved in a highly consistent manner, in which inter- and intra-individual variations are significantly reduced or eliminated, providing the physician and end user with a dosage form that is capable of providing far more reliable therapeutic effect.

We have also found that, since some of the bioadhesive carrier materials may swell extensively upon contact with a mucosal surface and thereby form gel structures, in some instances at least some of the active ingredient may be incorporated in-situ into a gel formed on top of the epithelia, so providing, at least in part, a sustained drug release.

Compositions of the invention may also have the advantage that they may be prepared using established pharmaceutical processing methods and employ materials that are approved for use in foods or pharmaceuticals or of like regulatory status.

Compositions of the invention may also have the advantage that they may be more efficacious than, be less toxic than, be more potent than, produce fewer side effects than, be more easily absorbed than, and/or have a better pharmacokinetic profile than, and/or have other useful pharmacological, physical, or chemical properties over, pharmaceutical compositions known in the prior art.

The invention is illustrated by way of the following examples, with reference to the accompanying figures in which:

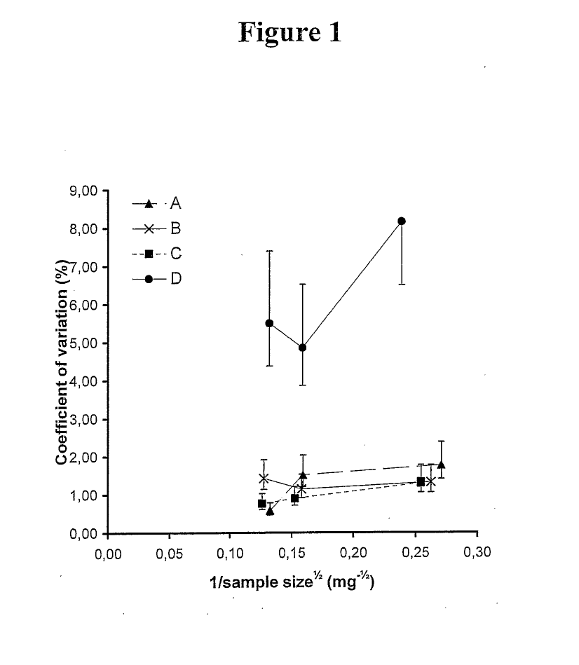

FIG. 1 shows plots of the coefficient of variation for the content of sodium salicylate in respect of the mean values obtained for samples extracted from various mixtures with sodium starch glycolate carrier particles, as a function of the inverse of the square root of the average size (in weight) of samples taken, in order to demonstrate the effect of carrier particle size on mixture homogeneity.

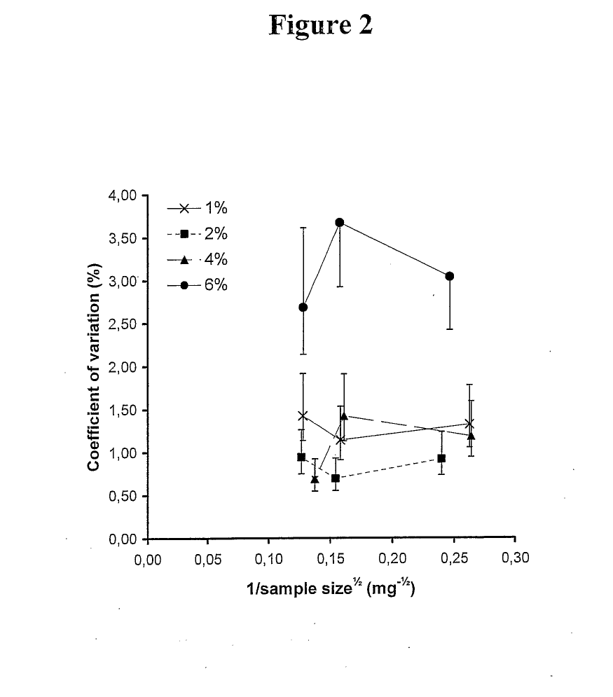

FIG. 2 shows similar plots to those of FIG. 1 in order to demonstrate the effect of active ingredient content on mixture homogeneity.

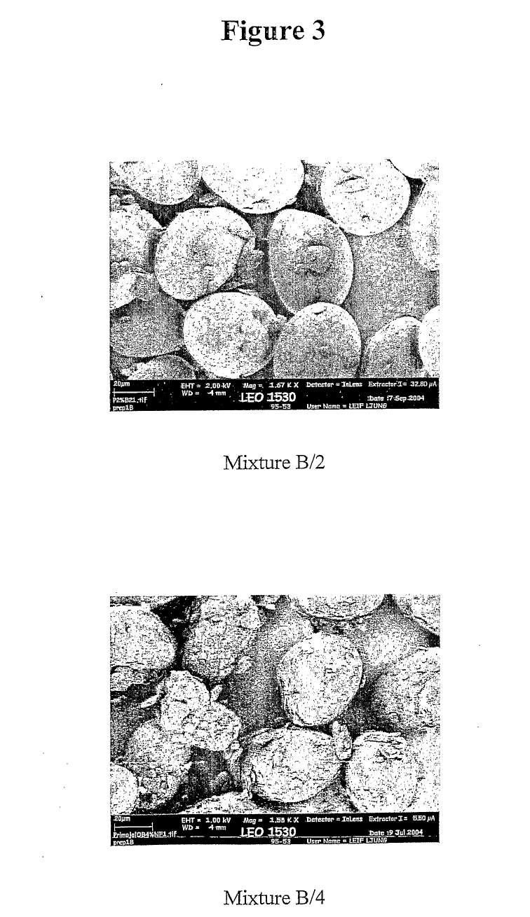

FIG. 3 shows scanning electron micrographs of two interactive mixtures of sodium salicylate and sodium starch glycolate.

### EXAMPLE 1

### Sodium Starch Glycolate Formulation

The aim of the present study was to investigate mixture homogeneity of formulations comprising sodium starch glycolate (Primojel®; DMV International BV, Netherlands) as carrier material and a model fine particulate drug compound, sodium salicylate (Sigma-Aldrich Sweden AB, Sweden).

### Materials

Carrier material particles were divided into various size fractions. The two finest size fractions (D and C) were obtained using an air classifier (100 MZR, Alpine, Germany); the two upper size fractions (B and A) were dry sieved (Retsch, Germany) to provide particles in the size range of between 32 and 45, and between 45 and 63 μm, respectively. The sieves were placed on a sieve shaker (Retsch RV 18412, Germany) for ten minutes and the procedure was repeated once more after cleaning in an aqueous solution containing alfa-amylase (Sigma-Aldrich Sweden AB, Sweden).

Sodium salicylate was milled in a mortar grinder (Retsch, Germany) for 10 minutes. The most coarse fraction was removed using the air classifier. All materials and mixtures were stored in desiccators at 18% RH.

Particle characteristics were measured and are shown in Table 1 below. Particle sizes are shown as median values by weight as measured by laser diffraction analysis (Sympatec Helos H0321, Germany). The size limits for which the cumulative amounts by weight from undersize distribution were equal to 10% and 90%, respectively, are shown in parentheses. Surface areas were measured by steady state permeametry (Johansson et al, Int. J Pharmaceutics, 163, 35 (1998)) or, in the case of sodium salicylate, by permearnetry using a Blaine apparatus (Kaye, Powder Technol., 1, 11 (1967)). The results are shown as the mean value from three measurements. The standard deviation is given in parentheses.

TABLE 1
Particle Sizes

| Material | Particle Size (μm) | Surface Area (m²/g) |
|---|---|---|
| A | 59.0 (49.3, 73.0) | 0.075 (0.002) |
| B | 44.8 (34.0, 58.0) | 0.092 (0.003) |
| C | 29.5 (21.2, 40.7) | 0.131 (0.001) |
| D | 16.2 (6.4, 24.6) | 0.236 (0.005) |
| Sodium salicylate | 3.17 (0.8, 10.8) | 1.77 (0.13) |

### Preparation of Mixtures

Mixtures were prepared in 50 g batches using a 250 mL glass jar (such that the vessel was not filled to more than one third of the total volume).

Mixtures containing 1% sodium salicylate were firstly prepared by adding 0.5 g of the model drug to 49.5 g of the four individual Primojel fractions. The glass jar was placed in a Turbula mixer (2 L W. A. Bachofen, Switzerland) at 67 rpm for 50 hours. If visible aggregates were still present thereafter, the mixing time was extended to 74 hours. Adhesion of drug to the container wall was regarded as insignificant. The small differences between the mixtures were considered enough to ensure reproducibility and no duplicates were prepared.

Mixtures containing higher drug amounts were also correspondingly prepared from carrier particle size fraction B.

Mixture characteristics are shown in Table 2 below. The percentage of sodium salicylate shown is the theoretical percentage. The exact percentage, according to empirical measurement, is given in parentheses. The surface area ratio is the ratio of projected surface area of sodium salicylate to the total external surface area of the relevant Primojel fraction, calculated according to the method described in Nyström et al, Int. J. Pharm., 10, 209 (1982). The ratio of particle sizes is a measure of the number of sodium salicylate particles divided by the number of particles of Primojel in the relevant fraction. The number of particles was calculated from size distributions by weight.

TABLE 2
Mixture Characteristics

| Size | | Surface | Ratio of particle |
|---|---|---|---|
| Fraction | % salicylate | area ratio | numbers by weight |
| A | 1 (0.99) | 5.91 | 920 |
| B | 1 (1.01) | 4.92 | 430 |
| C | 1 (1.01) | 3.46 | 110 |
| D | 1 (1.01) | 1.90 | 4.4 |
| B | 2 (2.01) | 9.84 | 860 |
| B | 4 (3.83) | 19.2 | 1700 |
| B | 6 (6.01) | 30.7 | 2700 |

### Mixture Homogeneity

Concentric cylinder powder thieves in three different sizes (15 mg (small), 40 mg (medium) and 60 mg (large)) were used to determine the mixture homogeneity. Thirty samples were taken at random positions with each powder thief for each mixture. The samples were dissolved in water and, after being vigorously shaken, were allowed to rest for 15 minutes. Primojel, which is not soluble in water, formed a sediment at the bottom of the test tube.

The UV absorption of the clear supernatant was measured at 295 nm (U1100, Hitachi, Japan). The percentage of sodium salicylate in the samples was calculated by means of a standard calibration curve.

Presented in Table 3 below, for each of the seven mixtures described above, are (a) the mean sample weight of 30 samples withdrawn from each mixture by the relevant thieves; and (b) the average percentage of sodium salicylate as measured spectrophotometrically. In both instances, standard deviations are presented in parentheses.

TABLE 3
Characteristics of Samples

| | Sample Weight | | | Average % of salicylate | | |
|---|---|---|---|---|---|---|
| Mixture | Small | Medium | Large | Small | Medium | Large |
| A/1 | 13.6 (2.6) | 39.4 (0.5) | 56.9 (3.4) | 0.98 (0.02) | 0.97 (0.02) | 0.97 (0.01) |
| B/1 | 14.5 (1.6) | 40.0 (3.0) | 61.6 (4.5) | 1.01 (0.01) | 1.00 (0.01) | 1.01 (0.01) |
| C/1 | 15.5 (1.0) | 42.9 (1.3) | 62.8 (1.1) | 0.97 (0.01) | 0.98 (0.01) | 0.98 (0.01) |
| D/1 | 17.5 (1.4) | 39.6 (1.8) | 57.5 (5.6) | 0.90 (0.07) | 0.95 (0.05) | 0.95 (0.05) |
| B/2 | 17.4 (1.7) | 42.0 (1.8) | 62.7 (6.1) | 1.81 (0.02) | 1.88 (0.01) | 1.90 (0.02) |
| B/4 | 14.4 (1.4) | 38.5 (0.8) | 53.1 (4.4) | 3.71 (0.04) | 3.75 (0.05) | 3.82 (0.03) |
| B/6 | 16.4 (1.5) | 40.5 (2.3) | 60.3 (4.4) | 5.70 (0.20) | 5.91 (0.22) | 5.87 (0.16) |

Mixture homogeneity is summarised in Table 4 below using the coefficient of variation (CV) as the prime measure (Williams, Powder Technology, 2, 13 (1968)). The standard deviations were assumed to follow a χ²-distribution and the confidence limits were calculated for the 96% probability level (Valentin, Chem. Eng., 5, CE99 (1967)).

TABLE 4
Summary of Experimental Results

| | Relative standard | | | | | |
|---|---|---|---|---|---|---|
| | deviation (%) | | | Confidence interval (%) | | |
| Mixture | Small | Medium | Large | Small | Medium | Large |
| A/1 | 1.77 | 1.51 | 0.58 | 1.41-2.38 | 1.21-2.02 | 0.46-0.78 |
| B/1 | 1.32 | 1.14 | 1.42 | 1.05-1.77 | 0.91-1.53 | 1.13-1.91 |
| C/1 | 1.29 | 0.89 | 0.76 | 1.03-1.73 | 0.71-1.20 | 0.61-1.02 |
| D/1 | 8.15 | 4.85 | 5.49 | 6.49-11.0 | 3.86-6.51 | 4.38-7.39 |
| B/2 | 0.92 | 0.69 | 0.94 | 0.73-1.23 | 0.55-0.93 | 0.75-1.26 |
| B/4 | 1.18 | 1.42 | 0.68 | 0.94-1.59 | 1.13-1.91 | 0.55-0.92 |
| B/6 | 3.04 | 3.67 | 2.68 | 2.42-4.08 | 2.92-4.93 | 2.14-3.61 |

The effects of carrier particle size, and drug content, on mixture homogeneity are shown in FIGS. 1 and 2 respectively. Scanning electron micrographs of mixtures B/2 and B/4 are shown in FIG. 3.

The results show that interactive mixtures of surprisingly good homogeneity may be prepared with carrier particles of a small size.

### EXAMPLE 2

### Desmopressin Powder Formulation

Desmopressin (99.93 mg, purity 95.6%) was dissolved in 100 mL of ethanol (99.5%) to a concentration of 0.955 mg/mL. 25 mL of the desmopressin solution was added to a round-bottomed flask with 10 g of pre-gelatinized starch (particle size less than 100 μm). The starch was wetted but not dissolved by the ethanol. The ethanol was then evaporated using a rotary evaporator until the starch powder, to which desmopressin was adhered, was dry and free flowing.

The theoretical concentration of desmopressin in the evaporated desmopressin/starch powder was 2.39 μg desmopressin per mg desmopressin/starch. Dose analysis of the powder showed a concentration of 2.25 μg desmopressin per mg desmopressin/starch.

### EXAMPLE 3

### Desmopressin Tablet Formulation

The dried powder of Example 2 was mixed with the following excipients: additional pre-gelatinized starch, mannitol, silicified microcrystalline cellulose and magnesium stearate. This mixture was direct compressed on a tablet press.

A target of 5 mg of desmopressin/starch per tablet was set. With a concentration of 2.25 μg desmopressin per mg desmopressin/starch, the tablets should have had an average content of 11.25 μg of desmopressin per tablet. Dose analysis of the tablets showed an average concentration of 10.86 μg desmopressin per tablet, with a relative standard deviation of 2.3%.

1. A pharmaceutical composition in the form of a homogeneous interactive mixture, which composition comprises a pharmacologically-effective amount of an active ingredient in the form of microparticles of a size between about 0.5 îm and about 10 îm, which particles are attached to the surfaces of larger carrier particles with a size range of between about 10 and about 100 îm.

2. A composition as claimed in claim 1, wherein the active ingredient is a peptide or a peptide hormone, a monoclonal antibody, an analgesic, an antiemetic or a sedative.

3. A composition as claimed in claim 2 wherein the active ingredient is sumatriptan, zolmitriptan, frovatriptan, dihydroergotamine, butorphanol, desmopressin, calcitonin-salmon, oxytocin, naferelin, buserelin, nicotine, vitamin B12, alprazolam, clonazepam, lorazepam, buprenorphine, nalbuphine, alfentanil, sufentanil, ramifentanil, granisetron, ramosetron, dolasetron, propofol, tadafinil, sildenafil, lobeline, deslorelin, vardenafil, insulin, glucagon, oxycodone, pumactant, apomorphine, lidocaine, dextromethorphane, ketamine, morphine, fentanyl, pramorelin, ondansteron, interferon alpha, interferon beta, scopolamine, vomeropherin, alprazolam, triazolam, midazolam, parathyroid hormone, growth hormone, GHRH, somatostatin, melatonin, a vaccine for H5n1 avian influenza, E coli, streptococcus A, influenza, parainfluenza, RSV, shigella, heliobacter pylori, versinia pestis, AIDS, rabies or periodontitis, an antiarthritic vaccine, Factor VIII, P-glucocerebrosidase human growth hormone, erythropoetin, interferon alfacon-1, interferon alfa-2b, peginterferon alfa-2b, interferon beta-1a, interferon beta-1b, interferon gamma-1b, granulocyte colony stimulating factor, pegfilgrastim, granulocyte-macrophage colony stimulating factor, interleukin-11, a recombinant form of interleukin-2, interleukin-1 receptor antagonist, infliximab or a mixture thereof.

4. A composition as claimed in claim 3 wherein the active ingredient is desmopressin, fentanyl, ketamine, buprenorphine or butorphanol.

5. A composition as claimed in claim 1, wherein the active ingredient particle size is between about 1 μm and about 8 μm.

6. A composition as claimed in claim 1 wherein the total amount of active ingredient that is present is in the range about 0.05 to about 5% by weight based upon the total weight of the composition.

7. A composition as claimed in claim 6, wherein the range is about 0.1 to about 1% by weight.

8. A composition as claimed in claim 1 wherein the carrier particles are bioadhesive and/or mucoadhesive in their nature.

9. A composition as claimed in claim 1 wherein the carrier particles consist essentially of bioadhesion and/or mucoadhesion promoting agent.

10. A composition as claimed in claim 9, wherein the bioadhesion and/or mucoadhesion promoting agent is a polymeric substance with a weight average molecular weight above 5,000.

11. A composition as claimed in claim 10, wherein the bioadhesion and/or mucoadhesion promoting agent is selected from a cellulose derivative, a starch derivative, an acrylic polymer, polyvinylpyrrolidone, polyethylene oxide, chitosan, a natural polymer, scleroglucan, xanthan gum, guar gum, poly co-(methylvinyl ether/maleic anhydride), crosscarmellose and mixtures thereof.

12. A composition as claimed in claim 11, wherein the bioadhesion and/or mucoadhesion promoting agent is selected from hydroxypropylmethyl cellulose, hydroxyethyl cellulose, hydroxypropyl cellulose, methyl cellulose, ethyl hydroxyethyl cellulose, carboxymethyl cellulose, modified cellulose gum, sodium carboxymethyl cellulose, moderately cross-linked starch, modified starch, sodium starch glycolate, carbomer or a derivative thereof, crosslinked polyvinylpyrrolidone, polyethylene oxide, chitosan, gelatin, sodium alginate, pectin, scleroglucan, xanthan gum, guar gum, poly co-(methylvinyl ether/maleic anhydride), crosscarmellose sodium and mixtures thereof.

13. A composition as claimed in claim 12, wherein the bioadhesion and/or mucoadhesion promoting agent is sodium starch glycolate or crosslinked polyvinylpyrrolidone.

14. A composition as claimed in claim 9, wherein the amount of bioadhesion and/or mucoadhesion promoting agent present is in the range of about 60% to about 99% by weight based upon the total weight of the composition.

15. A composition as claimed in claim 1 wherein the carrier particles are of a size of between about 15 and about 95 îm.

16. A composition as claimed in claim 15 wherein the size is between about 15 and about 80 îm.

17. A composition as claimed in claim 16, wherein the size is between about 20 and about 60 Tm.

18. A composition as claimed in claim 1, which further comprises a pharmaceutically acceptable surfactant or wetting agent.

19. A composition as claimed in claim 18, wherein the surfactant is sodium lauryl sulphate, a polysorbate, a bile acid salt or a mixture thereof, and/or is present in an amount of between about 0.3 and about 5% by weight based upon the total weight of the composition.

20. A composition as claimed in claim 1, which is essentially free of water.

21. A composition as claimed in claim 1, which is in the form of a powder.

22. A composition as claimed in claim 1, which is in the form of a tablet.

23. A composition as claimed in claim 1 which is suitable for administration to the nasal cavity.

24. A process for the preparation of a composition as defined in claim 1, which process comprises the step of dry mixing the carrier particles together with particles of active ingredient for a sufficient time to provide a homogeneous interactive mixture.

25. A process for the preparation of a composition as defined in claim 22, which comprises a process as claimed in claim 24, followed by compressing or compacting the resultant powder into tablet form.

26. (canceled)

27. A method of treatment of a disease, which comprises administration of a composition as defined in claim 1 to a patient in need of such treatment.

28. A method of administering an active ingredient to a patient, which comprises administration of a composition as defined in claim 1 to a mucosal surface in a patient in need of such administration.

29. A method of improving the uptake of an active ingredient across a mucosal surface in a patient, which comprises administration of a composition as defined in claim 1 to that mucosal surface.

30. A method of increasing the rate of absorption of an active ingredient across a mucosal surface in a patient, which comprises administration of a composition as defined in claim 1 to that mucosal surface.

31. A method as claimed in claim 28, wherein the mucosal surface is the nasal mucosa.

32. A method as claimed in claim 27, wherein the active ingredient is an analgesic, an antiemetic, a sedative, a peptide or a peptide hormone.

33. A method as claimed in claim 32 wherein the active ingredient is desmopressin, fentanyl, ketamine, buprenorphine or butorphanol.
